(19) United States
(12) Patent Application Publication
(10) Pub. No.: US 2021/0172012 A1
(43) Pub. Date: Jun. 10, 2021

(54) PREPARATION OF DNA SEQUENCING LIBRARIES FOR DETECTION OF DNA PATHOGENS IN PLASMA

(71) Applicant: ILLUMINA, INC., San Diego, CA (US)

(72) Inventors: Tong Liu, San Diego, CA (US); Fiona Kaper, San Diego, CA (US); Clifford Wang, San Diego, CA (US)

(73) Assignee: ILLUMINA, INC., San Diego, CA (US)

(21) Appl. No.: 17/109,348

(22) Filed: Dec. 2, 2020

Related U.S. Application Data
(60) Provisional application No. 62/943,459, filed on Dec. 4, 2019.

(51) Int. Cl. C12Q 1/6869; C12Q 1/6888

(52) U.S. Cl. CPC C12Q 1/6869; C12Q 2563/149; C12Q 2523/32; C12Q 1/6888

(57) ABSTRACT

The application provides an agnostic, shotgun nucleic acid sequencing-based method for the detection of pathogens in samples from human patients, animals, or plants. The method includes dehosting the sample of the nucleic acid molecules of host origin and provides for the detection of pathogens without prior knowledge of their genome sequences.

### CONTINUING APPLICATION DATA

This application claims the benefit of U.S. Provisional Application Ser. No. 62/943,459, filed Dec. 4, 2019, which is incorporated by reference herein.

### BACKGROUND

Currently, the detection of pathogens in samples from human patients, animals, or plants is commonly accomplished by antibody-based methods, polymerase chain reaction (PCR), or targeted nucleic acid capture followed by sequencing. Each of these approaches requires a targeting reagent, for example, an antibody or a DNA oligonucleotide, and thus requires prior knowledge of the pathogen. As a result, these methods can fail to detect previously undiscovered or otherwise ignored pathogens. Certainly, after a pathogen of interest is identified, targeted methods can be developed. Yet because new detection reagents would likely be required, any clinical detection or diagnostic test must be re-approved by regulatory agencies, increasing the cost and time to bring a product to market.

In contrast, an agnostic, shotgun nucleic acid sequencing approach can detect pathogens without prior knowledge of their genome sequences. With such an agnostic approach, nucleic acids are not enriched, amplified, or targeted based on the pathogen's genome sequence. Because pathogens are not detected according to their sequences, different reagents are not required for different pathogens. Thus, little, or no regulatory updates are necessary for the sample preparation and sequencing protocol, significantly decreasing the costs and time-to-market for clinical products.

The detection of pathogens by agnostic sequencing is challenging because samples usually contain an overwhelming amount of host nucleic acids. Because of the abundance of host nucleic acids, the sensitivity of detection is quite low. Without additional enrichment, in order to overcome this low sensitivity, a tremendous amount of sequencing is required. Since all nucleic acids in a sample, from both host and pathogen, are sequenced, the majority of sequencing reagents unnecessarily goes towards sequencing the host genome. This additional sequence burden can put many detection applications out of reach.

In order to increase the sensitivity of detection and reduce sequencing costs associated with agnostic, shotgun sequencing approaches, there is a need for improved methods of efficiently removing host DNA from samples and thus, enriching pathogen DNA.

### SUMMARY OF THE INVENTION

The present invention includes a sample preparation method that includes obtaining a host organism sample, removing intact cells from the host organism sample, and removing nucleic acid molecules of less than 1000 basepairs (bp) from the host organism sample to obtain a dehosted sample. In some aspects, the method further includes sequencing the nucleic acid molecules remaining in the dehosted sample. In some aspects, the method includes preparing a sequencing library from the nucleic acid molecules remaining in the dehosted sample and, in some aspects, further sequencing the nucleotide sequences of the sequencing library. In some aspects, the method further includes identifying pathogen sequences within the sequenced sequences.

The present invention includes a method of dehosting a sample obtained from a host organism, the method including removing intact cells from the host organism sample and removing nucleotide acid molecules of less than 1000 basepairs (bp) from the host organism sample to obtain a dehosted sample. In some aspects, the method further includes sequencing the nucleic acid molecules remaining in the dehosted sample. In some aspects, the method includes preparing a sequencing library from the nucleic acid molecules remaining in the dehosted sample and, in some aspects, further sequencing the nucleotide sequences of the sequencing library. In some aspects, the method further includes identifying pathogen sequences within the sequenced sequences.

The present invention includes a method of identifying pathogen nucleotide sequences in a sample obtained from a host organism, the method including removing intact cells from the host organism sample, removing nucleotide acid molecules of less than 1000 basepairs (bp) from the host organism sample to obtain a dehosted sample, preparing a sequencing library from the nucleic acid molecules remaining in the dehosted sample, sequencing the nucleotide sequences of the sequencing library, and identifying pathogen sequences within the sequenced sequences.

In some aspects of the methods described herein, the sequencing library is prepared by a transposon-based library preparation method. In some aspects, the transposon-based library preparation method includes NEXTERA transposons or NEXTERA bead-based transposons.

In some aspects of the methods described herein, sequencing is by high throughput sequencing.

In some aspects of the methods described herein, removing nucleotide acid molecules of less than 1000 basepairs (bp) from the host organism sample includes removing nucleic acid molecules of less than 600 bp from the host organism sample to obtain the dehosted sample.

In some aspects of the methods described herein, the method includes removing intact cells from the host organism sample by centrifugation.

In some aspects of the methods described herein, the method includes removing intact cells from the host organism sample by binding cell free nucleic acids to functionalized controlled pore glass (CPG) beads. In some aspects, the functionalized controlled pore glass (CPG) beads are functionalized with a copolymer of N-vinyl pyrrolidone (70%) and N-methyl-N-vinyl imidazolium chloride (30%).

In some aspects of the methods described herein, removing nucleotide acid molecules of less than 1000 bp from the host organism sample includes solid phase reversible immobilization (SPRI) beads under conditions favoring capture of nucleotide molecules of 1000 bp or greater.

In some aspects of the methods described herein, pathogen sequences include viral, bacterial, fungal, and/or parasitic sequences.

In some aspects of the methods described herein, pathogen sequences include a pathogen with a DNA genome.

In some aspects of the methods described herein, the host organism sample includes blood.

In some aspects of the methods described herein, the host organism sample includes plasma.

In some aspects of the methods described herein, the host includes a eukaryotic organism.

In some aspects of the methods described herein, the host includes an animal or plant.

In some aspects of the methods described herein, the host includes a mammal.

In some aspects of the methods described herein, the host includes a human.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### DEFINITIONS

The term “and/or” means one or all of the listed elements or a combination of any two or more of the listed elements.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the invention.

As used herein, the term “each,” when used in reference to a collection of items, is intended to identify an individual item in the collection but does not necessarily refer to every item in the collection unless the context clearly dictates otherwise.

The term “comprises,” and variations thereof, do not have a limiting meaning where these terms appear in the description and claims.

It is understood that wherever embodiments are described herein with the language “include,” “includes,” or “including,” and the like, otherwise analogous embodiments described in terms of “consisting of” and/or “consisting essentially of” are also provided.

Unless otherwise specified, “a,” “an,” “the,” and “at least one” are used interchangeably and mean one or more than one.

Also, herein, the recitations of numerical ranges by endpoints include all numbers subsumed within that range (for example, 1 to 5 includes 1, 1.5, 2, 2.75, 3, 3.80, 4, 5, etc.).

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

For any method disclosed herein that includes discrete steps, the steps may be conducted in any feasible order. And, as appropriate, any combination of two or more steps may be conducted simultaneously.

All headings are for the convenience of the reader and should not be used to limit the meaning of the text that follows the heading, unless so specified.

Reference throughout this specification to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments.

### BRIEF DESCRIPTION OF THE FIGURES

FIG. 1. Improved detection of pathogens in plasma is accomplished by size-selective DNA capture and transposon-based library preparation.

FIG. 2. Detection of λ virus spike-in (1000 copies/ml) in plasma. By employing optimized Solid Phase Reversible Immobilization (SPRI) size-selection and transposon concentrations, detection sensitivity of viral DNA was increased 10-fold.

FIG. 3. Electropherogram of plasma DNA size distribution showing approximately 95% of the DNA fragments in plasma are less than 600 bp. Short reads estimated using 400 bp insert length for virus, 170 for human, average weight of one DNA bp is 650 Da, average weight of one RNA base is 340 Da, 400 million reads per NextSeq.

FIG. 4. Electropherogram of plasma DNA size distribution when 84% of <600 bp DNA fragments are removed using Solid Phase Reversible Immobilization (SPRI) beads under conditions that strongly favor the capture of long DNA. Short reads estimated using 400 bp insert length for virus, 170 for human, average weight of one DNA bp is 650 Da, average weight of one RNA base is 340 Da, 400 million reads per NextSeq.

FIG. 5. Transposon-based methods are particularly suitable for preparation of Sequencing Libraries from plasma DNA.

FIG. 6. Sequencing experiments demonstrate that the efficiency of library generation drops significantly when DNA fragments are less than 1000 bp.

### DETAILED DESCRIPTION

While DNA sequencing can be used to detect pathogens and diagnose infectious diseases, the detection of pathogens by agnostic shotgun nucleic acid sequencing is challenging because samples contain a large, overwhelming amount of host nucleic acids. As all nucleic acids in the sample are sequenced, sequencing yields a vast majority of host sequences and a minority of pathogen sequences. Thus, the resultant sensitivity for pathogen detection is very low. The present invention provides improved methods for sample preparation and nucleic acid sequencing for the detection of pathogens in samples obtained from eukaryotic hosts.

The methods described herein include the dehosting of a sample of the nucleic acids of host origin. Such dehosting provides for the efficient removal of nucleic acids of host origin from the sample, providing for the enrichment of pathogen nucleic acids in the sample. Library preparation and DNA sequencing of such dehosted samples can then be undertaken to identify nucleic acids of pathogen origin. Without such dehosting, pathogen detection by unbiased sequencing has low sensitivity and is not feasible for the majority of clinical and industrial applications.

Currently, the detection of pathogens is commonly accomplished by antibody-based methods, polymerase chain reaction (PCR), or targeted nucleic acid capture followed by sequencing. Each of these approaches requires a targeting reagent, for example, an antibody or DNA oligonucleotide, and thus requires prior knowledge of the pathogen. As a result, these methods can fail to detect previously undiscovered or otherwise ignored pathogens. Certainly, after a pathogen of interest is identified, targeted methods can be developed. Yet because new detection reagents would likely be required, any clinical detection or diagnostic test must be re-approved by regulatory agencies, increasing the cost and time to bring a product to market.

In contrast, an agnostic, shotgun nucleic acid sequencing approach can detect pathogens without prior knowledge of their genome sequences. With such an agnostic approach, nucleic acids are not enriched, amplified, or targeted based on the pathogen's genome sequence. Because pathogens are not detected according to their sequences, different reagents are not required for different pathogens. However, the detection of pathogens by agnostic sequencing is challenging because a sample usually contains an overwhelming amount of host nucleic acids. Thus, to increase the sensitivity of detection and reduce sequencing costs for an agnostic, shotgun sequencing approach, the methods of the present invention efficiently remove host DNA from a sample.

For the methods described herein, a sample is obtained or provided. A sample may be a biological sample, including but not limited to, whole blood, blood serum, blood plasma, sweat, tears, urine, feces, sputum, cerebrospinal fluid, sperm, lymph, saliva, amniotic fluid, tissue biopsy, cell culture, swab, smear, or formalin-fixed paraffin-embedded (FFPE) sample. In some embodiments, a biological sample is a cell free plasma sample.

In some aspects, a sample may be an environmental sample, including but not limited, a food sample, a water sample, a soil sample, or an air sample, including, but not limited to, swabs, smear, or filtrates thereof.

A sample may be from a host organism. A host organism may be a eukaryotic organism, such as for example, an animal or plant. In some embodiments, a host organism is a mammal, including human hosts as well as non-human mammalian hosts.

For the methods described herein, intact cells may be removed from the sample. Intact cells may be removed from a sample by centrifugation or other cell separation methods. If using centrifugation, a low centrifugal force (e.g., 300×g) may be used so that host cells are removed from the sample and pathogens that are not inside host cells, such as, for example, mycoplasma, are not removed from the sample.

For the methods described herein, a sample may be “dehosted” of nucleic acids of host origin. Such dehosting involves the removal of nucleic acids of eukaryotic host origin, enriching the sample for nucleic acids of non-host, pathogen origin. Dehosting may be achieved by size selection for larger DNA fragments. In its natural state, eukaryotic nuclear DNA is not found as free linear strands. Rather, it is highly condensed and wrapped around histones in order to fit inside of the nucleus and take part in the formation of chromosomes. Histones are a family of basic proteins that associate with DNA in the nucleus, packaging and ordering the DNA into structural units called nucleosomes. Histone proteins are among the most highly conserved proteins in eukaryotes, emphasizing their important role in the biology of the nucleus (see, for example, Henneman et al., 2018, PLoS Genetics; 14 (9):e1007582). Histones are found in the nuclei of eukaryotic cells, but not in bacteria or viral genomes. In eukaryotes, octameric histone cores compact DNA by wrapping an approximately 150 bp unit twice around its surface, forming a nucleosome (Kornberg, 1974, Science; 184(4139):868-71). Because eukaryotic nuclear DNA is highly organized by coiling around histones to form nucleosome, circulating fragments of eukaryotic DNA outside of the nucleus tend to have a fairly uniform length of about 150 bp. Thus, removing smaller fragments from a cell free sample or isolating larger sized fragments from a cell free sample can effectively provide a sample that has been dehosted of nucleic acids of eukaryotic host origin.

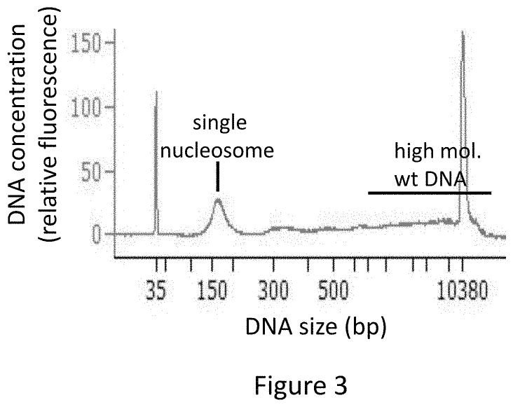

As shown in FIG. 3, cell-free DNA found in human plasma is dominated by shorter DNA fragments, with 95% or more of the DNA fragments being less than 600 bp. Since nearly all pathogen genomes are greater than 1 kb, one can dehost plasma prior to sequencing by selectively depleting these short fragments.

With removing smaller nucleic acid fragments from a cell free sample, fragments of about 1 kb or less, about 800 bp or less, about 600 bp or less, about 500 bp or less, about 400 bp or less, or about 200 bp or less in length may be removed from the sample. These nucleic acid fragments may be double stranded DNA fragments, single stranded DNA molecules, or RNA molecules. In some preferred embodiments, they are double stranded DNA fragments.

With isolating/purifying larger sized nucleic acid fragments from a cell free sample, fragments of about 200 bp or greater, about 400 bp or greater, about 600 bp or greater, about 800 bp or greater, or about 1 kb or greater may be isolated or purified from the sample. These nucleic acid fragments may be double stranded DNA molecules, single stranded DNA molecules, or RNA molecules. In some preferred embodiments, they are double stranded DNA fragments.

Any of a number of available technologies may be utilized for the enrichment of larger nucleic acid fragments, including, but not limited to size selection by electrophoresis followed by gel extraction, chromatography, or other solid phase extraction. Solid phase extraction methods include, but are not limited to, non-specifically and reversibly absorbing nucleic acids to silica beads (Boom et al., 1990, J. Clin Microbiol; 28(3):495-503) or carboxyl-coated paramagnetic particles, such as Solid Phase Reversible Immobilization (SPRI) Magnetic Beads (Beckman-Coulter's Agencourt AMPure XP beads; see DeAngelis et al., 1995, Nucleic Acids Res; 23(22):4742-3 and U.S. Pat. Nos. 5,705,628, 6,534,262, and 5,898,071.

For example, removing smaller nucleotide acid molecules from a host organism sample can be accomplished with the use of solid phase reversible immobilization (SPRI) beads under conditions favoring capture of nucleotide molecules of about 200 bp or greater, about 400 bp or greater, about 600 bp or greater, about 800 bp or greater, or about 1 kb or greater. The volume of SPIR beads to sample volume can be adjusted to provide for conditions that favor the capture of longer, nonhost nucleic acids. While a SPRI volume about 1.8 times (1.8×) that of the sample is typically used for the buffer exchange and cleanup of common PCR products, a volume of about 0.5× can be used to selectively capture primarily large DNA fragments, subsequently removing as much as 84% of host fragments <600 bp from human plasma DNA.

With the methods described herein, a sequencing library may then be prepared from the nucleic acid molecules remaining in a dehosted sample. Any of many established methods for preparing a sequencing library may be used. Library preparation may be for use with any of a variety of next generation sequencing platforms, such as for example, the sequencing by synthesis platform of ILLUMINA® or the ion semiconductor sequencing platform of ION TORRENT™. For example, established ligase-dependent methods or transposon-based methods may be used (Head et al, 2014, Biotechniques; 56(2):61) and numerous kits for making sequencing libraries by these methods are available commercially from a variety of vendors.

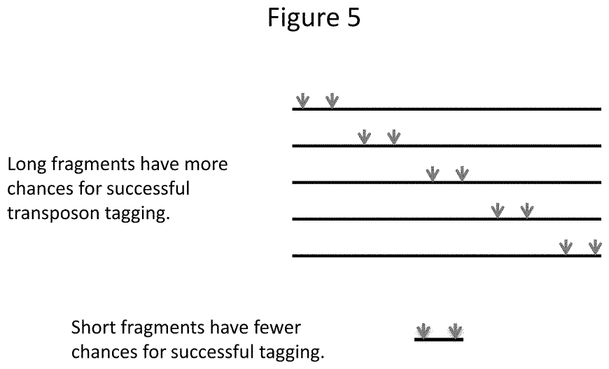

Transposon-based methods, which prepare DNA libraries by using a transposase enzyme to simultaneously fragment and tag DNA in a single-tube reaction termed “tagmentation,” are particularly suitable for pathogen detection in plasma DNA. First, transposon methods are faster and require fewer protocol steps than ligase-dependent methods, leading to shorter turnaround times for detection assays. Second, when transposons are used to tag DNA with sequencing adapters, the tagging and successful preparation of a sequencing library from long DNA fragments is favored over that of short fragments. Thus transposon-based library preparation can preferably enrich for larger non-host DNA fragments for sequencing. Thus, dehosting may be further enhanced by using transposon-based library preparation. Transposon based tagmentation methods may be solution based (see, for example, Adey et al., 2010, Genome Biol; 11(12):R119); Picelli et al., 2014, Genome Res; 24(12):2033; and Illumina® Nextera® DNA Library Prep Reference Guide, Document #15027987 v01, January 2016, WO 2010/048605; US 2012/0301925; and US 2013/0143774) or may utilize bead-immobilized transposomes conjugated directly to beads, such as magnetic-bead linked transposomes (BLT) (see, for example, Bruinsma et al, 2018, BMC Genomics; 19:722; and NEXTERA™ DNA Flex Library Prep Kit, Illumina, 2017; WO 2014/108810; and US 2018/0155709 A1). This is shown in FIG. 5.

With the methods described herein, the sequencing library representing the nucleic acid molecules remaining in the dehosted sample is then sequenced. Sequencing may be by any of a variety of known methodologies, including, but not limited to any of a variety high-throughput, next generation sequencing platforms, including, but not limited to, sequencing by synthesis, sequencing by ligation, nanopore sequencing, Sanger sequencing, and the like. In some embodiments, sequencing is performed using the sequencing by synthesis methodologies commercialized by ILLUMINA® as described in U.S. Patent Application Publication No. 2007/0166705, U.S. Patent Application Publication No. 2006/0188901, U.S. Pat. No. 7,057,026, Beijing Genomics Institute (BG) as described in Carnevali et al., 2012, J Comput Biol; 9(3):279-92 (doi: 10.1089/cmb.2011.0201. Epub 2011 Dec. 16), or the ion semiconductor sequencing methodologies of ION TORRENT™ as described in US 2009/0026082 A1; US 2009/0127589 A1; US 2010/0137143 A1; or US 2010/0282617 A1, each of which is incorporated herein by reference.

With the methods described herein, the resultant sequence information is then analyzed, and pathogen sequences identified by any of a variety of available methods, including, but not limited to, K-mer analysis and comparison against genome databases of known pathogens. Pathogens include, for example, viruses, bacteria, fungi, or parasites. In some aspects, a pathogen has a DNA genome, for example, a DNA virus. In some aspects, a pathogen has an RNA genome, for example, an RNA virus.

In same applications of the methods described herein, steps may be integrated, deleted, and/or combined.

While pathogens, such as viruses, may be present at very low concentrations in the original sample, dehosting the sample by the methods described herein can remove 99% of host DNA and increase sensitivity and reduce reagent costs by as much as 100-fold.

The disclosure includes kits for use in a method of dehosting a sample of eukaryotic host nucleic acids and/or identifying pathogen nucleotide sequences in a sample obtained from a eukaryotic host organism. A kit is any manufacture (e.g. a package or container) including at least one reagent for specifically of dehosting a sample of eukaryotic host nucleic acids and/or identifying pathogen nucleotide sequences in a sample obtained from a eukaryotic host organism. The kit may include instructions for use. The kit may be promoted, distributed, or sold as a unit for performing the methods of the present disclosure.

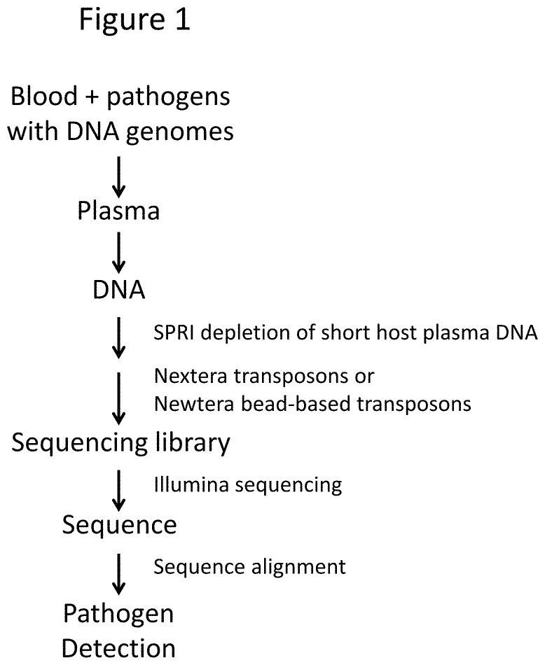

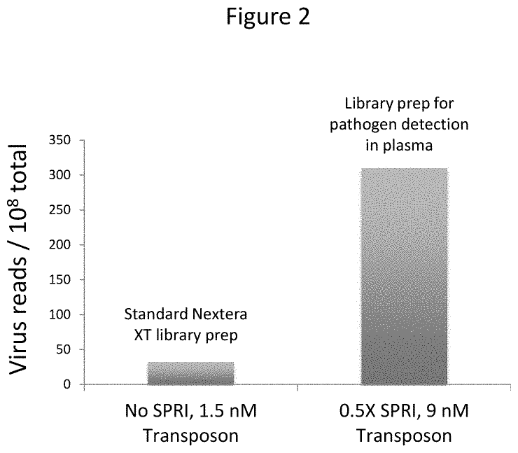

In one application of the method described herein improved detection of pathogens in plasma is accomplished by size-selective DNA capture and transposon-based library preparation (FIG. 1). By employing optimized SPRI size-selection and transposon concentrations, detection sensitivity of viral DNA was increased 10-fold. By employing optimized SPRI size-selection and transposon concentrations, detection sensitivity of viral DNA can be increased 10-fold (FIG. 2). As shown in FIG. 3, in human plasma, the majority of human DNA is present as short cell-free fragments. Approximately 95% of the DNA fragments in human plasma are less than 600 basepairs (bp) in length. Since nearly all pathogen genomes are greater than 1 kilobase (kb) in length, the methods described herein dehost plasma prior to the sequencing and detection of pathogen DNA genomes by selectively depleting a sample of these short fragments.

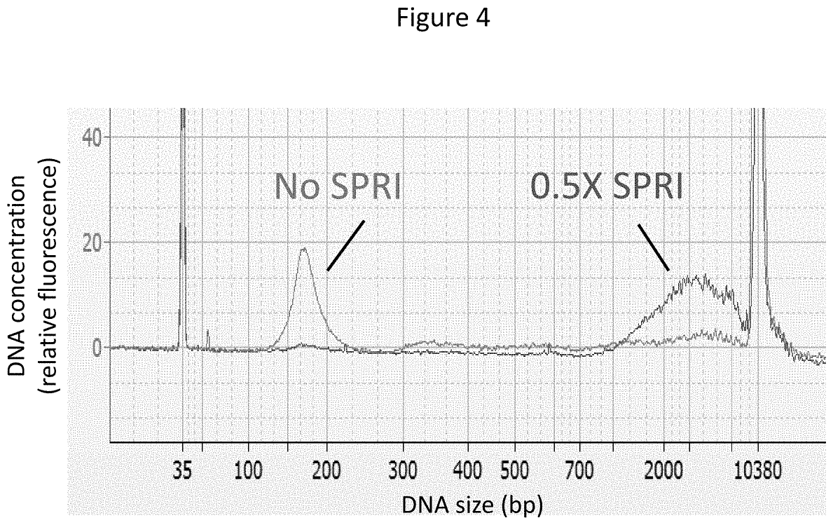

In some aspects, capturing long DNA and effectively removing shorter human DNA results in the enrichment of the sample for pathogen DNA. As shown in FIG. 4, 84% of DNA fragments <600 bp were removed using Solid Phase Reversible Immobilisation (SPRI) beads under conditions that strongly favor the capture of long DNA.

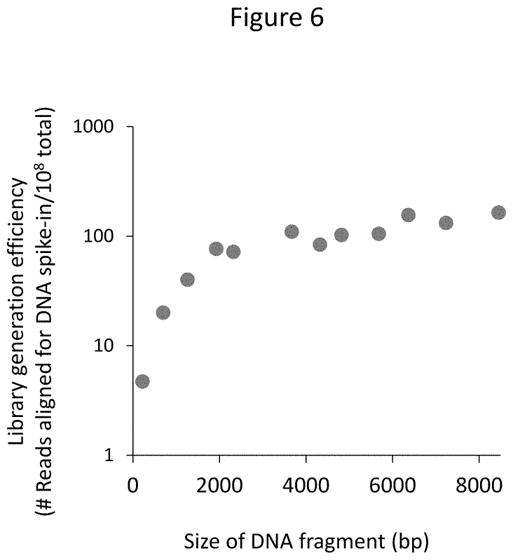

While any method to prepare Illumina sequencing libraries can be used for pathogen detection applications, transposon-based methods are particularly suitable for plasma DNA. Transposon methods are faster and require fewer protocol steps than ligase-dependent methods, leading to a shorter turn-around time for detection assays. When transposons in solution (Illumina Nextera) are used to tag DNA with sequencing adapters, the tagging of long DNA fragments is favored over short fragments. As shown in FIG. 5, long fragments have more chances for successful transposon tagging, while short fragments have fewer chances for successful tagging. Nextera or other transposon-based library prep methods thus effectively dehost plasma DNA samples by favoring larger DNA fragments. As shown in FIG. 6, sequencing experiments demonstrate that the efficiency of library generation drops significantly when DNA fragments are <1000 bp.

### DEFINITIONS

As used herein, the term “nucleic acid” is intended to be consistent with its use in the art and includes naturally occurring nucleic acids or functional analogs thereof. Particularly useful functional analogs are capable of hybridizing to a nucleic acid in a sequence specific fashion or capable of being used as a template for replication of a particular nucleotide sequence. Naturally occurring nucleic acids generally have a backbone containing phosphodiester bonds. An analog structure can have an alternate backbone linkage including any of a variety of those known in the art. Naturally occurring nucleic acids generally have a deoxyribose sugar (e.g. found in deoxyribonucleic acid (DNA)) or a ribose sugar (e.g. found in ribonucleic acid (RNA)). A nucleic acid can contain any of a variety of analogs of these sugar moieties that are known in the art. A nucleic acid can include native or non-native bases. In this regard, a native deoxyribonucleic acid can have one or more bases selected from the group consisting of adenine, thymine, cytosine or guanine and a ribonucleic acid can have one or more bases selected from the group consisting of uracil, adenine, cytosine or guanine. Useful non-native bases that can be included in a nucleic acid are known in the art. The term “template” and “target,” when used in reference to a nucleic acid, is intended as a semantic identifier for the nucleic acid in the context of a method or composition set forth herein and does not necessarily limit the structure or function of the nucleic acid beyond what is otherwise explicitly indicated.

As used herein, “amplify,” “amplifying” or “amplification reaction” and their derivatives, refer generally to any action or process whereby at least a portion of a nucleic acid molecule is replicated or copied into at least one additional nucleic acid molecule. The additional nucleic acid molecule optionally includes sequence that is substantially identical or substantially complementary to at least some portion of the target nucleic acid molecule. The target nucleic acid molecule can be single-stranded or double-stranded and the additional nucleic acid molecule can independently be single-stranded or double-stranded. Amplification optionally includes linear or exponential replication of a nucleic acid molecule. In some embodiments, such amplification can be performed using isothermal conditions; in other embodiments, such amplification can include thermocycling. In some embodiments, the amplification is a multiplex amplification that includes the simultaneous amplification of a plurality of target sequences in a single amplification reaction. In some embodiments, “amplification” includes amplification of at least some portion of DNA and RNA based nucleic acids alone, or in combination. The amplification reaction can include any of the amplification processes known to one of ordinary skill in the art. In some embodiments, the amplification reaction includes polymerase chain reaction (PCR).

As used herein, “amplification conditions” and its derivatives, generally refers to conditions suitable for amplifying one or more nucleic acid sequences. Such amplification can be linear or exponential. In some embodiments, the amplification conditions can include isothermal conditions or alternatively can include thermocyling conditions, or a combination of isothermal and thermocycling conditions. In some embodiments, the conditions suitable for amplifying one or more nucleic acid sequences include polymerase chain reaction (PCR) conditions. Typically, the amplification conditions refer to a reaction mixture that is sufficient to amplify nucleic acids such as one or more target sequences, or to amplify an amplified target sequence ligated to one or more adapters, e.g., an adapter-ligated amplified target sequence. Generally, the amplification conditions include a catalyst for amplification or for nucleic acid synthesis, for example a polymerase; a primer that possesses some degree of complementarity to the nucleic acid to be amplified; and nucleotides, such as deoxyribonucleotide triphosphates (dNTPs) to promote extension of the primer once hybridized to the nucleic acid. The amplification conditions can require hybridization or annealing of a primer to a nucleic acid, extension of the primer and a denaturing step in which the extended primer is separated from the nucleic acid sequence undergoing amplification. Typically, but not necessarily, amplification conditions can include thermocycling; in some embodiments, amplification conditions include a plurality of cycles where the steps of annealing, extending, and separating are repeated. Typically, the amplification conditions include cations such as Mg⁺⁺ or Mn⁺⁺ and can also include various modifiers of ionic strength.

The term “Next Generation Sequencing (NGS)” herein refers to sequencing methods that allow for massively parallel sequencing of clonally amplified molecules and of single nucleic acid molecules. Non-limiting examples of NGS include sequencing-by-synthesis using reversible dye terminators, and sequencing-by-ligation.

As used herein, the term “polymerase chain reaction” (PCR) refers to the method of K. B. Mullis U.S. Pat. Nos. 4,683,195 and 4,683,202, which describes a method for increasing the concentration of a segment of a polynucleotide of interest in a mixture of genomic DNA without cloning or purification. This process for amplifying the polynucleotide of interest consists of introducing a large excess of two oligonucleotide primers to the DNA mixture containing the desired polynucleotide of interest, followed by a series of thermal cycling in the presence of a DNA polymerase. The two primers are complementary to their respective strands of the double-stranded polynucleotide of interest. The mixture is denatured at a higher temperature first and the primers are then annealed to complementary sequences within the polynucleotide of interest molecule. Following annealing, the primers are extended with a polymerase to form a new pair of complementary strands. The steps of denaturation, primer annealing, and polymerase extension can be repeated many times (referred to as thermocycling) to obtain a high concentration of an amplified segment of the desired polynucleotide of interest. The length of the amplified segment of the desired polynucleotide of interest (amplicon) is determined by the relative positions of the primers with respect to each other, and therefore, this length is a controllable parameter. By virtue of repeating the process, the method is referred to as the “polymerase chain reaction” (hereinafter “PCR”). Because the desired amplified segments of the polynucleotide of interest become the predominant nucleic acid sequences (in terms of concentration) in the mixture, they are said to be “PCR amplified.” In a modification to the method discussed above, the target nucleic acid molecules can be PCR amplified using a plurality of different primer pairs, in some cases, one or more primer pairs per target nucleic acid molecule of interest, thereby forming a multiplex PCR reaction.

As used herein, the term “primer” and its derivatives refer generally to any polynucleotide that can hybridize to a target sequence of interest. Typically, the primer functions as a substrate onto which nucleotides can be polymerized by a polymerase; in some embodiments, however, the primer can become incorporated into the synthesized nucleic acid strand and provide a site to which another primer can hybridize to prime synthesis of a new strand that is complementary to the synthesized nucleic acid molecule. The primer can include any combination of nucleotides or analogs thereof. In some embodiments, the primer is a single-stranded oligonucleotide or polynucleotide. The terms “polynucleotide” and “oligonucleotide” are used interchangeably herein to refer to a polymeric form of nucleotides of any length, and may comprise ribonucleotides, deoxyribonucleotides, analogs thereof, or mixtures thereof. The terms should be understood to include, as equivalents, analogs of either DNA or RNA made from nucleotide analogs and to be applicable to single stranded (such as sense or antisense) and double-stranded polynucleotides. The term as used herein also encompasses cDNA, that is complementary or copy DNA produced from an RNA template, for example by the action of reverse transcriptase. This term refers only to the primary structure of the molecule. Thus, the term includes triple-, double- and single-stranded deoxyribonucleic acid (“DNA”), as well as triple-, double- and single-stranded ribonucleic acid (“RNA”).

The term “flowcell” as used herein refers to a chamber comprising a solid surface across which one or more fluid reagents can be flowed. Examples of flowcells and related fluidic systems and detection platforms that can be readily used in the methods of the present disclosure are described, for example, in Bentley et al., Nature 456:53-59 (2008), WO 04/018497; U.S. Pat. No. 7,057,026; WO 91/06678; WO 07/123744; U.S. Pat. Nos. 7,329,492; 7,211,414; 7,315,019; 7,405,281, and US 2008/0108082.

As used herein, the term “amplicon,” when used in reference to a nucleic acid, means the product of copying the nucleic acid, wherein the product has a nucleotide sequence that is the same as or complementary to at least a portion of the nucleotide sequence of the nucleic acid. An amplicon can be produced by any of a variety of amplification methods that use the nucleic acid, or an amplicon thereof, as a template including, for example, PCR, rolling circle amplification (RCA), ligation extension, or ligation chain reaction. An amplicon can be a nucleic acid molecule having a single copy of a particular nucleotide sequence (e.g. a PCR product) or multiple copies of the nucleotide sequence (e.g. a concatameric product of RCA). A first amplicon of a target nucleic acid is typically a complimentary copy. Subsequent amplicons are copies that are created, after generation of the first amplicon, from the target nucleic acid or from the first amplicon. A subsequent amplicon can have a sequence that is substantially complementary to the target nucleic acid or substantially identical to the target nucleic acid.

As used herein, the term “array” refers to a population of sites that can be differentiated from each other according to relative location. Different molecules that are at different sites of an array can be differentiated from each other according to the locations of the sites in the array. An individual site of an array can include one or more molecules of a particular type. For example, a site can include a single target nucleic acid molecule having a particular sequence or a site can include several nucleic acid molecules having the same sequence (and/or complementary sequence, thereof). The sites of an array can be different features located on the same substrate. Exemplary features include without limitation, wells in a substrate, beads (or other particles) in or on a substrate, projections from a substrate, ridges on a substrate or channels in a substrate. The sites of an array can be separate substrates each bearing a different molecule. Different molecules attached to separate substrates can be identified according to the locations of the substrates on a surface to which the substrates are associated or according to the locations of the substrates in a liquid or gel. Exemplary arrays in which separate substrates are located on a surface include, without limitation, those having beads in wells.

The term “sensitivity” as used herein is equal to the number of true positives divided by the sum of true positives and false negatives.

The term “specificity” as used herein is equal to the number of true negatives divided by the sum of true negatives and false positives.

As used herein, “providing” in the context of a composition, an article, a nucleic acid, or a nucleus means making the composition, article, nucleic acid, or nucleus, purchasing the composition, article, nucleic acid, or nucleus, or otherwise obtaining the compound, composition, article, or nucleus.

The invention is defined in the claims. However, below is provided a non-exhaustive list of non-limiting embodiments. Any one or more of the features of these embodiments may be combined with any one or more features of another example, embodiment, or aspect described herein.

Embodiment 1 is a sample preparation method comprising: obtaining a host organism sample; removing intact cells from the host organism sample; removing nucleic acid molecules of less than 1000 basepairs (bp) from the host organism sample to obtain a dehosted sample.

Embodiment 2 is a method of dehosting a sample obtained from a host organism, the method comprising: removing intact cells from the host organism sample; removing nucleotide acid molecules of less than 1000 basepairs (bp) from the host organism sample to obtain a dehosted sample.

Embodiment 3 is the method of embodiment 1 or 2, further comprising sequencing the nucleic acid molecules remaining in the dehosted sample.

Embodiment 4 is the method of embodiment 1 or 2, further comprising preparing a sequencing library from the nucleic acid molecules remaining in the dehosted sample.

Embodiment 5 is the method of embodiment 4, further comprising sequencing the nucleotide sequences of the sequencing library.

Embodiment 6 is the method of embodiment 3 or embodiment 5, further comprising identifying pathogen sequences within the sequenced sequences.

Embodiment 7 is a method of identifying pathogen nucleotide sequences in a sample obtained from a host organism, the method comprising: removing intact cells from the host organism sample; removing nucleotide acid molecules of less than 1000 basepairs (bp) from the host organism sample to obtain a dehosted sample; preparing a sequencing library from the nucleic acid molecules remaining in the dehosted sample; sequencing the nucleotide sequences of the sequencing library; and identifying pathogen sequences within the sequenced sequences.

Embodiment 8 is the method of embodiment 4 or embodiment 7, wherein the sequencing library is prepared by a transposon-based library preparation method.

Embodiment 9 is the method of embodiment 8, wherein the transposon-based library preparation method comprises NEXTERA transposons or NEXTERA bead-based transposons.

Embodiment 10 is the method of any one of embodiments 3, 5, or 7 to 9, wherein sequencing is by high throughput sequencing.

Embodiment 11 is the method of any one of embodiments 1 to 10, comprising removing nucleic acid molecules of less than 600 bp from the host organism sample to obtain the dehosted sample.

Embodiment 12 is the method of any one of embodiments 1 to 11, wherein removing intact cells from the host organism sample comprises centrifugation.

Embodiment 13 is the method of claim any one of embodiments 1 to 12, wherein removing intact cells from the host organism sample comprises binding cell free nucleic acids to functionalized controlled pore glass (CPG) beads.

Embodiment 14 is the method of embodiment 13, wherein the functionalized controlled pore glass (CPG) beads are functionalized with a copolymer of N-vinyl pyrrolidone (70%) and N-methyl-N′-vinyl imidazolium chloride (30%).

Embodiment 15 is the method of any one of embodiments 1 to 14, wherein removing nucleotide acid molecules of less than 1000 bp from the host organism sample comprises solid phase reversible immobilization (SPRI) beads under conditions favoring capture of nucleotide molecules of 1000 bp or greater.

Embodiment 16 is the method of any one of embodiments 6 to 15, wherein the pathogen sequences comprise viral, bacterial, fungal, and/or parasitic sequence.

Embodiment 17 is the method of any one of embodiment 6 to 16, wherein the pathogen sequences comprise a pathogen with a DNA genome.

Embodiment 18 is the method of any one of embodiments 1 to 17, wherein the host organism sample comprises blood.

Embodiment 19 is the method of any one of embodiments 1 to 17, wherein the host organism sample comprises plasma.

Embodiment 20 is the method of any one of embodiments 1 to 19, wherein the host comprises a eukaryotic organism.

Embodiment 21 is the method of any one of embodiments 1 to 20, wherein the host comprises an animal or plant.

Embodiment 22 is the method of any one of embodiments 1 to 20, wherein the host comprises a mammal.

Embodiment 23 is the method of any one of embodiments 1 to 22, wherein the host comprises a human.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

### Preparation of DNA Sequencing Libraries for Detection of DNA Pathogens in Plasma

This example details a sample preparation strategy for the sequence detection of pathogens with DNA genomes (including, but not limited to, DNA viruses, bacteria, fungi, and parasites) in plasma. Improved detection of pathogens in plasma is accomplished by size-selective DNA capture and transposon-based library preparation. An overall schematic of the sample preparation methodology is shown in FIG. 1.

As shown in FIG. 3, in human plasma, the overwhelming majority of human DNA is present as short cell-free fragments. 95% or more of these DNA fragments are less than 600 bp. Since nearly all pathogen genomes are greater than 1 kb, one can dehost plasma prior to sequencing detection of pathogen DNA genomes by selectively depleting these short fragments. Dehosting is achieved by size selection for large DNA fragments and enhanced further by using transposon-based library preparation. By capturing long DNA, one can effectively remove shorter human DNA and enrich the sample for pathogen DNA.

One method for depleting short fragments is the use of Solid Phase Reversible Immobilization (SPRI) beads under conditions that strongly favor the capture of long DNA. While a SPRI volume 1.8 times (1.8×) that of the sample is typically used for the buffer exchange and cleanup of common PCR products, a 0.5× volume was found to selectively capture primarily large DNA fragments, subsequently removing as much as 84% of host fragments <600 bp from human plasma DNA.

With this example, 84% of <600 bp DNA fragments were removed using SPRI beads under conditions that strongly favor the capture of long DNA. See FIG. 4.

While any established method to prepare sequencing libraries can be used for pathogen detection applications, transposon-based methods are particularly suitable for pathogen detection in plasma DNA. First, transposon methods are faster and require fewer protocol steps than ligase-dependent methods, leading to shorter turnaround times for detection assays. Second, when transposons in solution (Illumina NEXTERA) are used to tag DNA with sequencing adapters, the tagging of long DNA fragments is favored over short fragments. Thus transposon-based library prep can preferably select and sequence DNA from larger fragments. Long fragments have more chances for successful transposon tagging/short fragments have fewer chances for successful tagging. As shown in FIG. 5, in experiments employing transposons in solution (Illumina NEXTERA), the efficiency of library generation was significantly higher for DNA fragments greater than 1 kb. NEXTERA or other transposon-based library preparation methods contribute inherently to dehosting plasma DNA samples favoring larger DNA fragments. As shown in FIG. 6, sequencing experiments demonstrate that the efficiency of library generation drops significantly when DNA fragments are <1000 bp.

To detect pathogens (in particular, those with DNA genomes) in the blood, one first prepares plasma and removes cells by centrifugation or other cell separation methods. If using centrifugation, a low centrifugal force (e.g., 300×g) is used so that host cells are removed and pathogens (those not inside cells, e.g., mycoplasma) are not. From the remaining plasma, one extracts cell-free DNA, which will also include pathogen DNA. From this cell-free DNA, using size selection or other methods, DNA is enriched for pathogen DNA. This DNA is then converted to a sequencing library by transposon or other molecular biology techniques. The library is then sequenced, and pathogen sequences are identified.

By combining optimized SPRI (0.5×) capture with an optimized concentration of transposon (9 nM NEXTERA transposon), pathogen detection sensitivity was increased by 10-fold compared to standard methods. Other variations of the invention can further improve detection sensitivity, decrease the time of sample prep, and simplify the protocol. In one variation of this method, one can also use transposons attached to solid beads (i.e., Illumina NEXTERA). In another variation of the method, host DNA first can be removed directly from blood or plasma by using functionalized controlled pore glass (CPG) beads that bind cell-free DNA, but not whole cells (e.g., bacteria and parasites) or viruses. One example of such beads are CPG beads functionalized with a copolymer of N-vinyl pyrrolidone (70%) and N-methyl-N′-vinylimidazolium chloride (30%).

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A sample preparation method comprising:
obtaining a host organism sample;
removing intact cells from the host organism sample;
removing nucleic acid molecules of less than 1000 basepairs (bp) from the host organism sample to obtain a dehosted sample.

2. A method of dehosting a sample obtained from a host organism, the method comprising:
removing intact cells from the host organism sample;
removing nucleotide acid molecules of less than 1000 basepairs (bp) from the host organism sample to obtain a dehosted sample.

3. The method of claim 1 further comprising sequencing the nucleic acid molecules remaining in the dehosted sample.

4. The method of claim 1 further comprising preparing a sequencing library from the nucleic acid molecules remaining in the dehosted sample.

5. The method of claim 4 further comprising sequencing the nucleotide sequences of the sequencing library.

6. The method of claim 3 further comprising identifying pathogen sequences within the sequenced sequences.

7. A method of identifying pathogen nucleotide sequences in a sample obtained from a host organism, the method comprising:
removing intact cells from the host organism sample;
removing nucleotide acid molecules of less than 1000 basepairs (bp) from the host organism sample to obtain a dehosted sample;
preparing a sequencing library from the nucleic acid molecules remaining in the dehosted sample;
sequencing the nucleotide sequences of the sequencing library; and
identifying pathogen sequences within the sequenced sequences.

8. The method of claim 7, wherein the sequencing library is prepared by a transposon-based library preparation method.

9. The method of claim 8, wherein the transposon-based library preparation method comprises NEXTERA transposons or NEXTERA bead-based transposons.

10. The method of claim 3, wherein sequencing is by high throughput sequencing.

11. The method of e of claim 1, comprising removing nucleic acid molecules of less than 600 bp from the host organism sample to obtain the dehosted sample.

12. The method of claim 1, wherein removing intact cells from the host organism sample comprises centrifugation.

13. The method of claim 1, wherein removing intact cells from the host organism sample comprises binding cell free nucleic acids to functionalized controlled pore glass (CPG) beads.

14. The method of claim 13, wherein the functionalized controlled pore glass (CPG) beads are functionalized with a copolymer of N-vinyl pyrrolidone (70%) and N-methyl-N-vinyl imidazolium chloride (30%).

15. The method of 1, wherein removing nucleotide acid molecules of less than 1000 bp from the host organism sample comprises solid phase reversible immobilization (SPRI) beads under conditions favoring capture of nucleotide molecules of 1000 bp or greater.

16. The method of claim 6, wherein the pathogen sequences comprise viral, bacterial, fungal, and/or parasitic sequence.

17. The method of claim 6, wherein the pathogen sequences comprise a pathogen with a DNA genome.

18. The method of claim 1, wherein the host organism sample comprises blood.

19. The method of claim 1, wherein the host organism sample comprises plasma.

20. The method of claim 1, wherein the host comprises a eukaryotic organism.

21. The method of claim 20, wherein the host comprises an animal or plant.

22. The method of claim 20, wherein the host comprises a mammal.

23. The method of claim 22, wherein the host comprises a human.
